(19) United States
(12) Patent Application Publication
(10) Pub. No.: US 2019/0000410 A1
(43) Pub. Date: Jan. 3, 2019

(54) X-RAY CT APPARATUS AND MEDICAL INFORMATION PROCESSING APPARATUS

(71) Applicant: CANON MEDICAL SYSTEMS CORPORATION, Otawara-shi (JP)

(72) Inventors: Takashi TANAKA, Nasushiobara (JP); Shinsuke Tsukagoshi, Nasushiobara (JP); Masaharu Tsuyuki, Nasushiobara (JP)

(73) Assignee: CANON MEDICAL SYSTEMS CORPORATION, Otawara-shi (JP)

(21) Appl. No.: 16/023,691

(22) Filed: Jun. 29, 2018

(30) Foreign Application Priority Data
Jun. 30, 2017 (JP) 2017-129014
Jun. 27, 2018 (JP) 2018-122209

(51) Int. Cl. A61B 6/00; A61B 6/03; G06T 7/00; G16H 30/40; G06N 99/00

(52) U.S. Cl. CPC A61B 6/545; A61B 6/032; G06T 7/0012; A61B 6/481; G06T 2207/30004; G16H 30/40; G06N 99/005; G06T 2207/10081; A61B 6/46

(57) ABSTRACT

According to one embodiment, an X-ray CT apparatus includes processing circuitry. The processing circuitry acquires examination information including a plurality of imaging conditions corresponding to an examination performed on an object, estimates an examination code corresponding to the examination based on the examination information, and associates the examination code with the examination information.

### CROSS-REFERENCE TO RELATED APPLICATIONS

This application claims the benefit of priority of Japanese Patent Application No. 2017-129014, filed Jun. 30, 2017, and Japanese Patent Application No. 2018-122209, filed Jun. 27, 2018, the entire contents of which are incorporated herein by reference.

### FIELD

Embodiments described herein relate generally to an X-ray CT apparatus and a medical information processing apparatus.

### BACKGROUND

Examples of modalities for imaging an object and performing image diagnosis include a magnetic resonance imaging (MRI) apparatus, a nuclear medical diagnostic apparatus such as a single photon emission computed tomography (SPECT) device and a positron emission tomography (PET) apparatus, an X-ray computed tomography (CT) apparatus, an ultrasonic diagnostic apparatus, and the like. These modalities execute examination based on examination order information, for example.

In recent years, examination order information may include examination code which is code value indicating examination information such as information on a body form of the object, an imaging mode, an imaged region, and the like. When the examination code is included in the examination order information, the user can confirm the examination information indicated by the examination code before executing the examination.

Further, when the examination is executed as instructed by the examination order information, the examination code included in the examination order information can be added to the record of the executed examination (hereinafter referred to as “examination record”). When the examination code is added to the examination record, it is very convenient for the user because the examination record can easily be accumulated and managed according to the contents of the examination.

However, the examination order information does not include any examination code in many cases. When the examination code is not included in the examination order information, the user has to manually add the examination code to the examination record (the record of the executed examination) after the examination is ended. Even if the examination code is included in the examination order information, the user is forced to manually add the examination code to the examination record after the examination is ended, when an examination different from the examination instructed by the examination order information has been executed.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing an example of an X-ray CT apparatus including a console device as an example of a medical information apparatus according to the present embodiment;

FIG. 2 is a block diagram illustrating the functions of processor of the processing circuitry;

FIG. 3 is an explanatory diagram showing an example of an examination code table;

FIG. 4 is a flowchart showing an example of a procedure for supporting so as to easily associate an examination code with examination information of the executed examination, by the processor of the medical information processing apparatus such as the processing circuitry;

FIG. 5 is a subroutine flowchart showing an example of a procedure of the examination code estimation process executed by the processor of the medical information processing apparatus such as the processing circuit in step S6 of FIG. 4;

FIG. 6 is an explanatory diagram showing an example of a list of the estimated examination codes; and

FIG. 7 is an explanatory diagram showing an example of an image for notifying information that the estimated code and the order code are different.

### DETAILED DESCRIPTION

Hereinbelow, a description will be given of an X-ray CT apparatus and a medical information processing apparatus according to embodiments of the present invention with reference to the drawings.

In general, according to one embodiment, an X-ray CT apparatus includes processing circuitry. The processing circuitry acquires examination information including a plurality of imaging conditions corresponding to an examination performed on an object, estimates an examination code corresponding to the examination based on the examination information, and associates the examination code with the examination information.

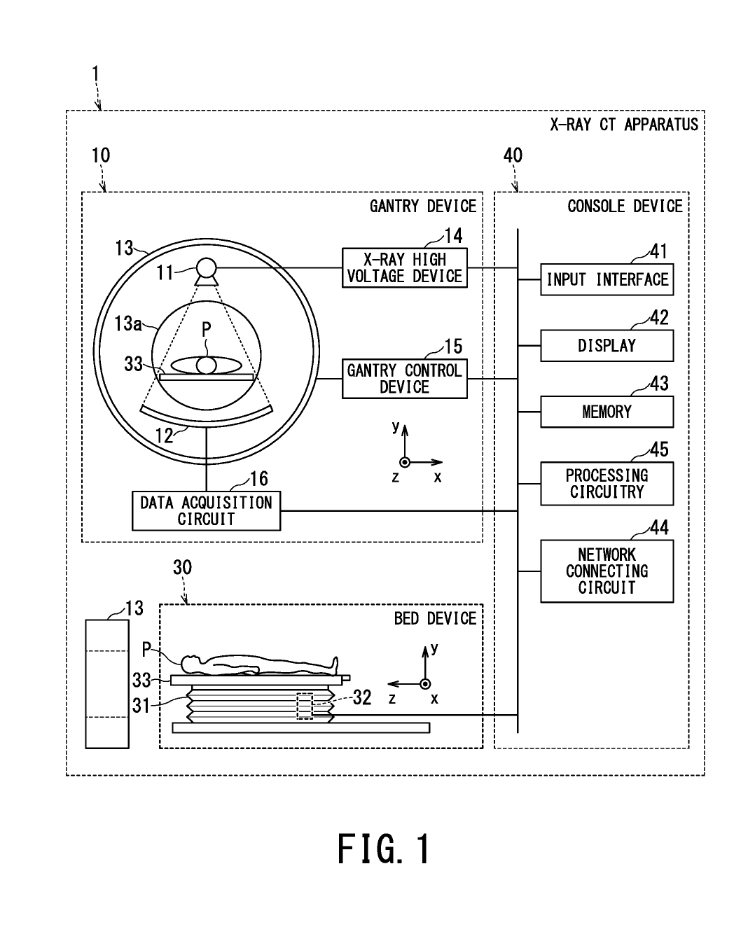

FIG. 1 is a block diagram showing an example of the X-ray CT apparatus 1 including the console device 40 as an example of a medical information apparatus according to the present embodiment.

The X-ray CT apparatus 1 includes a gantry device 10, a bed device 30, and a console device 40.

The X-ray CT apparatus 1 may be configured as any one of different types, such as a so-called third generation CT apparatus, i.e., an Rotate/Rotate type in which an X-ray tube and an X-ray detector integrally rotate about an object, or as a so-called fourth generation CT apparatus, i.e., an Stationary/Rotate type or an Nutate/Rotate type in which multiple detecting elements are circularly arrayed and only the X-ray tube rotates about the object. In the following, an example of adopting the third generation Rotate/Rotate type as the X-ray CT apparatus 1 according to the present embodiment is explained.

The gantry device 10 includes an X-ray generator 11, an X-ray detector 12, a rotating frame 13 having an opening 13 a in which an imaging region resides, an X-ray high voltage device 14, a gantry control device 15, and a DAS (Data Acquisition System) 16.

The X-ray generator 11 consists of an X-ray tube (vacuum tube) that is applied a high voltage from, for example, the X-ray high voltage device 14 and irradiates thermoelectrons from a cathode (filament) to an anode (target).

The X-ray generator according to this embodiment is applicable to a single tube type X-ray CT apparatus, and also applicable to a so-called multi-tube type X-ray CT apparatus in which a plurality of pairs of an X-ray tube and an X-ray detector is mounted on a rotating ring. Further, the X-ray source of the X-ray generator 11 is not limited to the X-ray tube. For example, when the X-ray CT apparatus is configured as a so called fifth generation type, the X-ray source of the X-ray generator 11, instead of the X-ray tube, can be used which includes a focus coil for focusing the electron beam generated from the electron gun, a deflection coil for electromagnetically deflecting the electron beam, and the target ring enclosing a half circumference of the object P and generating the X-rays by collided with the deflected electron beam.

The X-ray detector 12 has, for example, a plurality of X-ray detecting element arrays in which a plurality of X-ray detecting elements are arranged in the channel direction along one circular arc around the focal point of the X-ray tube as the center. The X-ray detector 12 has a structure in which the plurality of X-ray detection element arrays in the channel direction is arrayed in a slice direction. The X-ray detector 12 detects X-rays that have been irradiated from the X-ray generator 11 and passed through an object (such as a patient) P and outputs an electric signal according to the detected X-ray amounts to the DAS 16.

The X-ray detector 12 is, for example, an indirect conversion type detector having a grid, a scintillator array, and an optical sensor array. The scintillator array has a plurality of scintillators, and each scintillator has a scintillator crystal that outputs light with an amount of photons corresponding to the amount of incident X-rays. The grid is disposed on the X-ray incident side of the scintillator array and has an X-ray shielding plate having a function of absorbing scattered X-rays. The optical sensor array has a function of converting the light from the scintillator into an electric signal corresponding to the amount of the light from the scintillator, and has, for example, an optical sensor such as a photomultiplier tube.

The X-ray detector 12 may be a direct conversion type detector having a semiconductor element for converting incident X-rays into electric signals.

The rotating frame 13 holds together the X-ray generator 11, the X-ray detector 12, and the DAS 16, and rotates around the opening 13a of the central portion. In the present embodiment, the direction parallel to the rotation center axis of the rotating frame 13 is defined as the z-axis direction, the normal direction of the installation surface of the gantry device 10 is defined as the y-axis direction, and the direction parallel to the installation surface is defined as the x-axis direction (See FIG. 1).

The X-ray high voltage device 14 has an electric circuit including a transformer and a rectifier, and includes a high voltage generator having a function of generating a high voltage to be applied to the X-ray tube of the X-ray generator 11, and an X-ray controller for controlling the output voltage according to the X-rays irradiated by the X-ray tube. The high voltage generating device may be a transformer type or an inverter type.

The gantry control device 15 has a processor and a memory, and a driving mechanism such as a motor and an actuator. The gantry control device 15 receives an input signal from an input interface attached to the console device 40 or the gantry device 10, and controls the gantry device 10. For example, the gantry control device 15 performs rotation control of the rotating frame 13 in response to the input signal, tilt control of the gantry device 10, and driving control of the bed device 30 and the tabletop 33.

The DAS 16 includes at least an amplifier that performs amplification processing on electric signals output from each X-ray detecting element of the X-ray detector 12 and an A/D converter that converts an electric signal into a digital signal, and generates detection data (pure raw data). The detection data generated by the DAS 16 is transferred to the console device 40.

The bed device 30 is a device for placing and moving the object P to be scanned, and includes a base 31, a bed driving device 32, and a tabletop 33.

The base 31 is a casing that supports the tabletop 33 movably in the vertical direction (y direction). The bed driving device 32 is a motor or an actuator that moves the tabletop 33 on which the object P is placed in the longitudinal direction (z direction) of the tabletop 33. A tabletop 33 is a plate on which the object P is placed.

The bed driving device 32 may move the tabletop 33 alone or move the tabletop 33 together with the base 31 of the bed device 30. In the case where the present invention can be applied to a standing CT (an X-ray CT apparatus adapted to a standing object), the patient movement mechanism may be moved instead of the tabletop 33. Further, in the case of imaging involving a relative change in the positional relationship between the imaging system of the gantry device 10 and the tabletop 33, such as a helical scan and a positioning scan, the relative change of the positional relationship may be performed by driving the tabletop 33, or the gantry device 10, or by a combination thereof.

The console device 40 as an example of the medical information processing apparatus has an input interface 41, a display 42, a memory 43, a network connecting circuit 44, and a processing circuitry 45. The console device 40 will be described below as executing a plurality of functions with a single console, but a plurality of functions may be executed by different consoles.

The input interface 41 receives various input operations from the user, converts the received input operations into electric signals, and outputs them to the processing circuitry 45. For example, the input interface 41 accepts collection conditions for collecting projection data, reconstruction conditions for reconstructing a CT image, image processing conditions for generating a post-processing image from a CT image, and the like from the user. The input interface 41 may be a mouse, a keyboard, a track ball, a switch, a button, a joystick, a ten key, and the like.

The display 42 displays various kinds of information. For example, the display 42 outputs a medical image (CT image) generated by the processing circuitry 45, a GUI (Graphical User Interface) for receiving various operations from the user. The display 42 may be a liquid crystal display, a CRT (Cathode Ray Tube) display, an OLED (Organic Light Emitting Diode) display, and the like.

The memory 43 has a configuration including a processor-readable recording medium such as a semiconductor memory element, e.g., a RAM (Random Access Memory) or a flash memory, a hard disk, an optical disk, and the like. The memory 43 stores, for example, projection data and reconstructed image data.

The memory 43 may store a table (hereinafter referred to as an examination code table) 61 in which the examination code is associated in advance for each type of examination information. The processing circuitry 45 estimates the examination code corresponding to the examination actually performed (executed examination) using the examination code table 61. It should be noted that the examination code table 61 can be stored in an external storage circuit connected to the X-ray CT apparatus 1 via a network and used by the processing circuitry 45. Further, a part or all of the programs and data in the recording medium of the memory 43 may be downloaded by communication via the network.

The network connecting circuit 44 implements various information communication protocols according to the network. The network connecting circuit 44 connects the X-ray CT apparatus 1 and other devices in accordance with these various protocols. For this connection, electrical connection via an electronic network or the like can be applied. The network refers to a general information communication network using telecommunications technology and includes not only a wireless/wired LAN hospital backbone local area network (LAN) and the Internet network, but also a telephone communication network, an optical fiber communication network, a cable communication network, a satellite communication network, and other networks.

The processing circuitry 45 is a processor configured to execute, by reading out and executing the program stored in the memory 41, a procedure for supporting so as to easily associate the examination code with the examination information of the executed examination. The processing circuitry 45 also controls the entire operation of the X-ray CT apparatus 1 according to the electric signals that are corresponding to the input operations to the input interface 41 and are output therefrom.

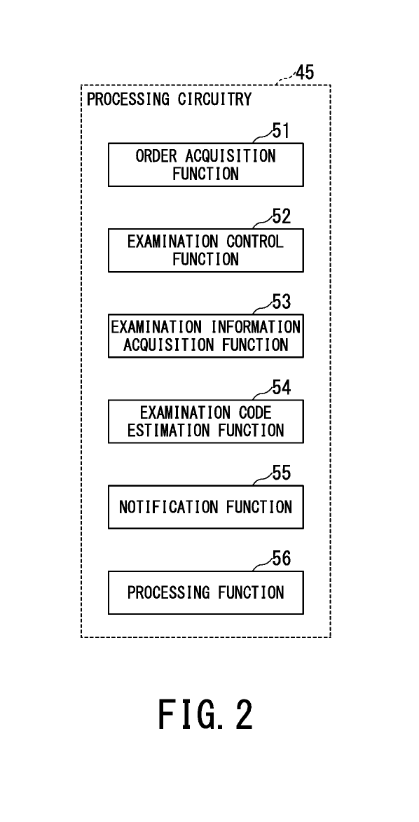

FIG. 2 is a block diagram illustrating the functions of processor of the processing circuitry 45.

As shown in FIG. 2, the processor of processing circuitry 45 implements the order acquisition function 51, the examination control function 52, the examination information acquisition function 53, the examination code estimation function 54, the notification function 55, and the processing function 56. Each of these functions is stored in the memory 43 in the form of a program.

Though an example will be described in the present embodiment in which each function 51-56 is implemented by the processing circuitry 45 of the console device 40 as an example of the medical information processing apparatus, some or all of these functions 51-56 of the medical information processing apparatus may be implemented by an external device having at least a processor and a memory circuit such as a server installed in a hospital, a cloud console, a workstation or the like connected to the X-ray CT apparatus 1.

The order acquisition function 51 acquires the examination order. The examination order is reservation information of the examination, and can be acquired from a hospital information system (HIS), a radiology information system (RIS), or the like via the network.

The examination control function 52 sets imaging conditions and the like based on the examination order, and executes examination of the object P.

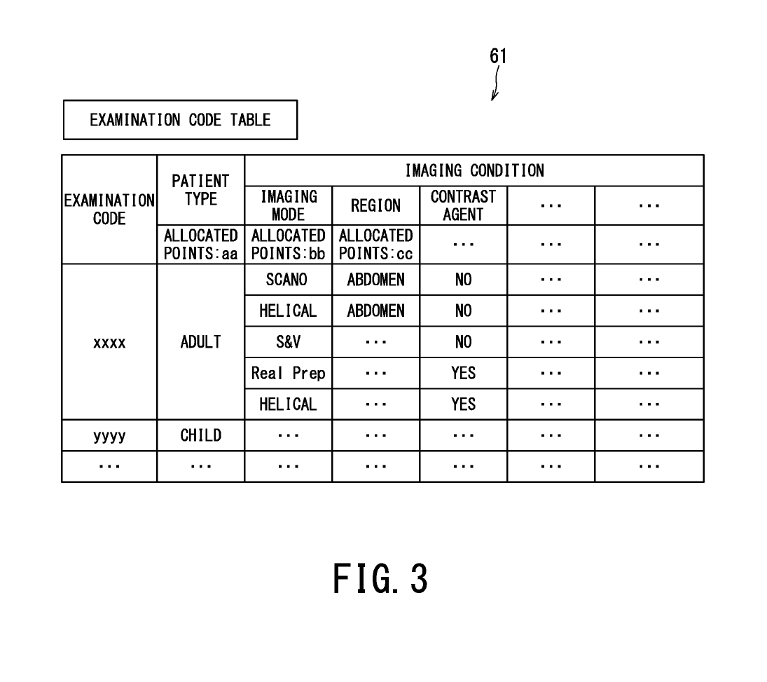

The examination information acquisition function 53 acquires from the gantry device 10 the examination information included in the record of the examination executed on the object P. The examination record includes the examination information including information (including the patient type) of the object P, the imaging conditions, and the like, of the actually executed examination. The imaging conditions included in the examination information include information such as examination time, tube current, tube voltage, and the like, in addition to the items of the imaging condition shown in FIG. 3.

The examination code estimation function 54 estimates the examination code corresponding to the executed examination based on the examination information.

For example, the examination code estimation function 54 may be able to use the results of machine learning such as deep learning. In this case, the examination code estimation function 54 may include a learning model based on an artificial neural network, for example. For example, the learning model performs machine learning such as deep learning beforehand with supervised learning using training data consisting of the examination information as an input object and the examination code corresponding to the examination information as a desired output value, and stores parameters after completion of the learning in memory 43 or an external storage circuit or the like on the network. In this case, by using the learned parameters, the examination code estimation function 54 can estimate the examination code corresponding to the examination by using the examination information. The examination code estimation function 54 may obtain the similarity (likelihood) of the examination code corresponding to the examination, may output one examination code having the highest similarity, or output examination codes having the similarities greater than or equal to a predetermined threshold value.

The examination code estimation function 54 may estimate the examination code corresponding to the executed examination by using the examination information included in the examination record (record of the examination actually performed), based on the examination code table 61 stored in the memory 43 or an external storage on the network.

Described below is an example in which the examination code estimation function 54 estimates the examination code based on the examination code table 61.

FIG. 3 is an explanatory diagram showing the example of the examination code table.

The examination code table 61 is a table in which each of the examination codes are associated in advance with corresponding each of the types of the examination information. The examination information includes the patient type and the imaging condition. The patient type may include information such as whether the object P is an adult or a child, information such as the age and physique of the object P, and the like. The imaging condition may include information such as the imaging mode, the imaged region (the imaged portion of the object P), the information on presence or absence of contrast agent, and contrast injection timing.

For example, in the example shown in FIG. 3, the examination information associated with the examination code “xxxx” includes the information indicates that the patient type is “adult”, the target part of the imaging is “abdominal”, the “helical” imaging is performed after the “scano” imaging is performed, and after the “S & V (Scan and View)” imaging (imaging a thin slice by one rotation scan) is performed the contrast agent is injected and the “Real Prep” imaging (imaging in which the imaging timing is set according to the arrival time of the contrast agent) is performed, and then the “helical” imaging is performed.

When there are many variations in the imaging method such as X-ray CT imaging, and when a plurality of imaging conditions are included in one examination, it is very complicated and difficult for the user to assign the proper examination code for each combination of the complicated imaging conditions. The X-ray CT apparatus 1 according to the present embodiment can easily estimate the examination code corresponding to the examination based on the examination information, even for the examination including a plurality of imaging conditions.

In the examination code table 61, similarity points may be previously allocated for each item of the examination information (see FIG. 3). In this case, the examination code estimation function 54 calculate similarity by comparing the examination information included in the examination record with the examination information corresponding to each of the plurality of examination codes after the examination is executed, and finds an examination code having the highest similarity score.

Further, information on the exposure dose may be associated with the examination code in advance in the examination code table 61.

The examination code estimation function 54 may extract an examination code in which the similarity score is equal to or greater than a predetermined threshold value. In this case, the examination code estimation function 54 displays a list 71 of the extracted examination codes (hereinafter referred to as a list of the estimated examination codes) on the display 42, and estimates one examination code specified by the user via the input interface 41 as the examination code corresponding to the executed examination.

When the examination code is included in the examination order and when the examination code estimated by the examination code estimation function 54 differs from the examination code included in the examination order, the notification function 55 notifies the user that the examination code estimated by the examination code estimation function 54 is different from the examination code included in the examination order.

The processing function 56 associates the examination code estimated by the examination code estimation function 54 with the examination information, and stores the examination code in, for example, the memory 43 or the image server. At this time, the processing function 56 may associate the examination information with the examination code by adding an examination code as additional information to the examination record corresponding to the examination information.

Next, an example of the operation of the X-ray CT apparatus 1 and the medical information processing apparatus according to the present embodiment will be described.

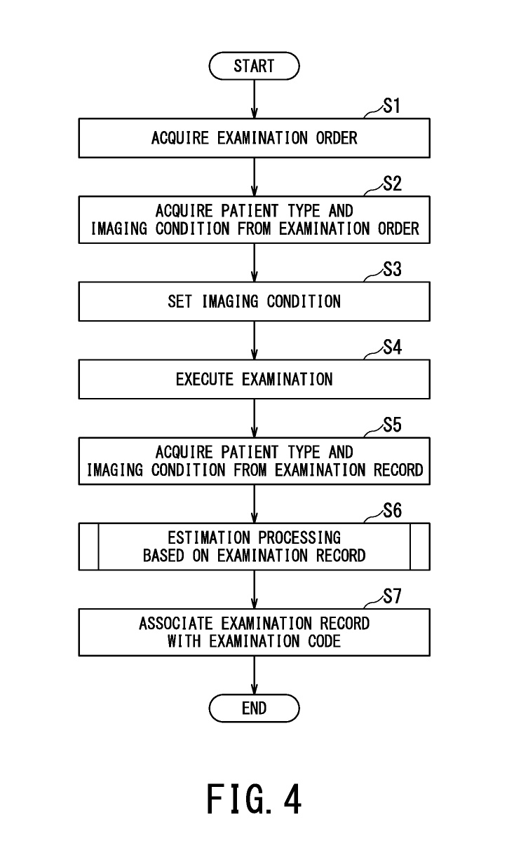

FIG. 4 is a flowchart showing an example of a procedure for supporting so as to easily associate the examination code with the examination information of the executed examination, by the processor of the medical information processing apparatus such as the processing circuitry 45. In FIG. 4, a reference character with “S” followed by a number denotes each step of the flowchart.

First, in step S1, the order acquisition function 51 acquires the examination order from HIS, RIS, and the like, via the network.

Next, in step S2, the examination control function 52 acquires the patient type and the imaging condition from the examination order.

Next, in step S3, the examination control function 52 sets the imaging conditions and the like based on the information acquired from the examination order. The examination control function 52 may automatically set the imaging conditions and the like based on the examination order, or, the examination control function 52 may display the contents of the examination order on the display 42 and then set the imaging conditions and the like according to the correction instruction via the input interface 41 given by the user checking the displayed contents of the examination order. Next, in step S4, the examination control function 52 executes the examination of the object P based on the set imaging conditions.

Next, in step S5, the examination information acquisition function 53 acquires the record of the examination executed on the object P from the gantry device 10 and acquires the examination information such as the patient type and the imaging condition from the examination record. When the medical information processing apparatus is an external device independent of the X-ray CT apparatus 1, the examination information acquisition function 53 may not acquire the examination record from the gantry device 10 but may acquire it from an image server or the like connected via the network.

The examination information acquisition function 53 performs the image processing on the medical image acquired by executing the examination, thereby automatically estimating the imaged region in the executed examination. As this kind of estimation method for estimating the imaged region, various kinds of methods such as an image processing method regarding usual alignment like a pattern matching method, a method using anatomical characteristic points (Anatomical Landmarks), and the like are well known, and any one of these methods can be applied.

When using the anatomical characteristic points, the examination information acquisition function 53 detects a portion such as an organ based on the anatomical characteristic points (Anatomical Landmarks) in the three-dimensional medical image data (volume data). Specifically, the examination information acquisition function 53 extracts anatomical characteristic points included in the volume data using, for example, a supervised machine learning algorithm.

Here, the supervised machine learning algorithm is constructed using a plurality of supervisory images in which correct anatomical characteristic points are manually arranged, by using decision trees or the like. The anatomical characteristic points are the points indicating features of a specific bone, organ, blood vessel, nerve, lumen, or the like.

That is, the examination information acquisition function 53 can detect bones, organs, blood vessels, nerves, lumens, and the like included in the volume data by detecting the anatomical characteristic points of the corresponding specific organs, bones, and the like. In addition, the examination information acquisition function 53 can also detect the positions of the head, neck, chest, abdomen, foot, etc. included in the volume data by detecting the characteristic feature points of the human body. More specifically, the examination information acquisition function 53 extracts anatomical characteristic points from the voxel values included in the volume data. Then, the examination information acquisition function 53 compares the three-dimensional position of the anatomical characteristic point in the information of the textbook and the like with the position of the characteristic point extracted from the volume data, thereby removing inaccurate characteristic points and optimizing the positions of the characteristic points extracted from the volume data. As a result, the examination information acquisition function 53 can detect each portion of the object P included in the volume data.

Next, in step S6, the examination code estimation function 54 executes a process of estimating the examination code corresponding to the examination based on the examination code table 61 stored in the memory 43 or an external storage circuit on the network or the like, by using the examination information acquired by the examination information acquisition function 53 from the examination record.

Next, in step S7, the processing function 56 stores the estimated examination code in association with the examination information, for example, in the memory 43 or an image server.

According to the above procedure, it is possible to support so as to easily associate the examination code with the examination information of the executed examination.

The processing circuitry 45 of the console device 40 as an example of the medical information processing apparatus according to the present embodiment can automatically estimate the examination code corresponding to the examination, after actual examination is executed, by using the examination information included in the examination record, based on the examination code table 61. Therefore, even when the examination code is not included in the examination order, the examination record can be easily classified into the appropriate examination code, so that management of the examination record becomes quite easy.

Hence, according to the medical information processing apparatus and the X-ray CT apparatus 1 according to the present embodiment, the examination information can be associated with an examination code according to the imaging conditions, and therefore, it is easy to manage for each imaging condition. For example, it is possible to easily search an examination code with a high use frequency for a predetermined examination purpose. Also, examination records can be easily classified into appropriate examination codes. Hence, when the radiation dose information is associated with the examination code in the examination code table 61, the exposure dose can be managed very easily.

Next, a procedure for estimating the examination code corresponding to the examination based on the examination code table 61 using the examination information acquired from the examination record will be explained.

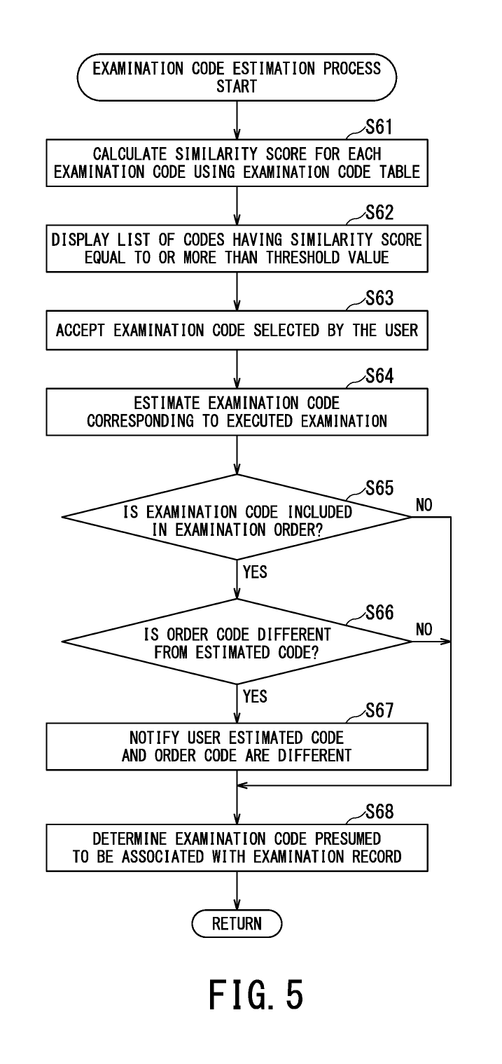

FIG. 5 is a subroutine flowchart showing an example of the procedure of the examination code estimation process executed by the processor of the medical information processing apparatus such as the processing circuit 45 in step S6 of FIG. 4. In FIG. 5, a reference character with “S” followed by a number denotes each step of the flowchart.

In step S61, the examination code estimation function 54 calculates the similarity score for each examination code using the examination information acquired by the examination information acquisition function 53 from the examination record.

Next, in step S62, the examination code estimation function 54 extracts an examination code in which the similarity score is equal to or larger than the predetermined threshold value, and displays the list 71 of the extracted examination codes on the display 42.

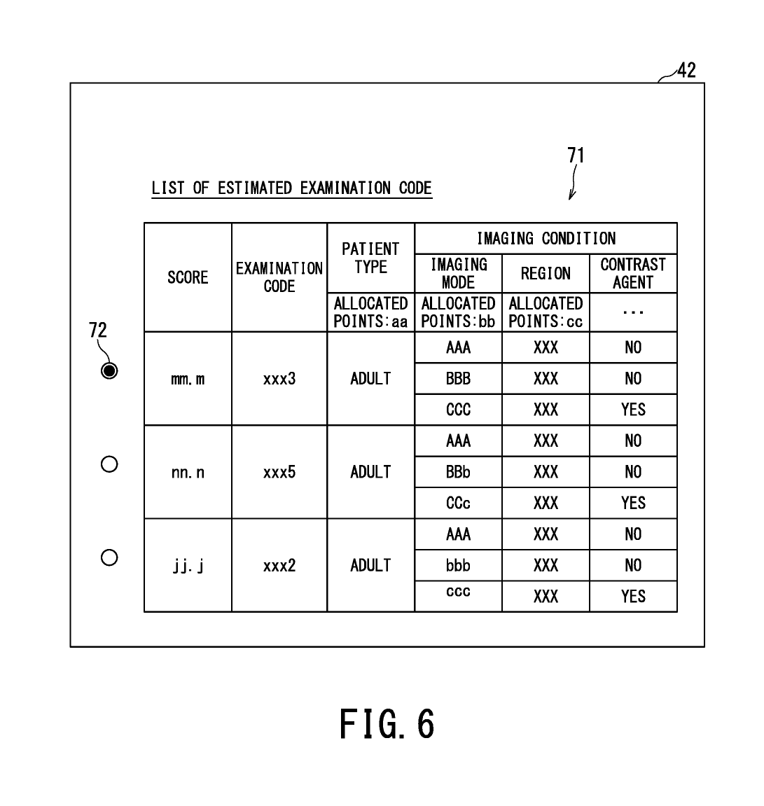

FIG. 6 is an explanatory diagram showing an example of the list 71 of the estimated examination codes.

As shown in FIG. 6, in the list 71 of the estimated examination codes, the examination codes whose similarity score are equal to or more than the predetermined threshold value is displayed in a list. In the list 71 of the estimated examination codes, for example, a button 72 for accepting an alternative selection by the user is displayed.

Next, in step S63, the examination code estimation function 54 accepts, for example, one examination code designated by the user via the input interface 41 using the button 72.

Next, in step S64, the examination code estimation function 54 estimates the examination code designated by the user as the examination code corresponding to the examination.

The examination code estimation function 54 may estimate the examination code having the highest similarity score as the examination code corresponding to examination in step S64. In this case, steps S62-63 are omitted, and the information on the examination code having the highest similarity score is displayed on the display 42 in a manner similar to the list 71 of the estimated examination codes. By accepting a confirmation instruction by the user, the examination code of the highest score may be estimated as the examination code corresponding to the examination.

Next, in step S65, the notification function 55 determines whether the examination code is included in the examination order. When the examination code is included in the examination order, the process proceeds to step S66. When the examination order is not included in the examination order, then the process proceeds to step S68.

Next, in step S66, the notification function 55 determines whether the examination code included in the examination order (hereinafter referred to as the order code) is different from the examination code estimated by the examination code estimation function 54 in step S64 (hereinafter referred to as the estimated code). When they are different, the flow proceeds to step S67, and when they are the same, the flow proceeds to step S68.

Next, in step S67, the notification function 55 notifies the user that the estimated code and the order code are different. As the notifying means, in addition to the display on the display 42, sounds via a speaker (not shown), a buzzer output, or the like can be used.

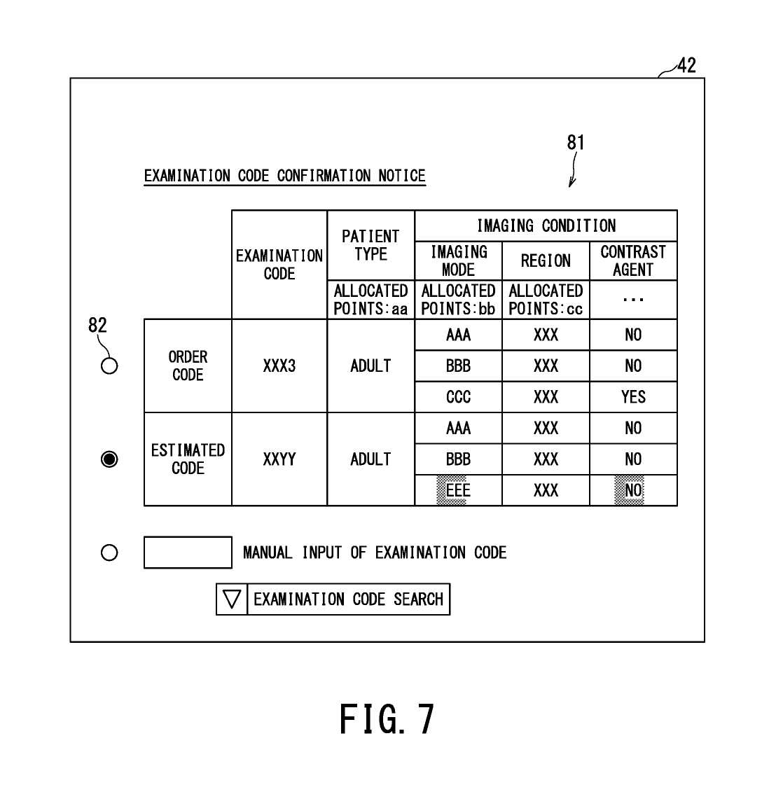

FIG. 7 is an explanatory diagram showing an example of an image for notifying information that the estimated code and the order code are different (hereinafter referred to as examination code confirmation notification image 81).

The notification function 55 notifies that the order code and the estimated code are different by displaying the examination code confirmation notification image 81 on the display 42. The examination code confirmation notification image 81 shown in FIG. 7 includes the examination information corresponding to the order code and the examination information corresponding to the estimated code. The notification function 55 may compare them and highlight the different items and notify the user (see hatching in FIG. 7). In the examination code confirmation notification image 81, for example, a button 82 for accepting alternative selection by the user is displayed. In addition, the examination code confirmation notification image 81 may accept input of the examination code manually by the user, and may also provide a search function of the examination code to support a manual input of the examination code (see the lower part of FIG. 7).

Then, in step S68, the examination code estimation function 54 determines the examination code presumed to be associated with the examination information, and proceeds to step S7 in FIG. 4.

For example, when proceeding from step S65 or step S66 to step S68, the examination code estimation function 54 determines the examination code estimated in step S64 as the examination code presumed to be associated with the examination information. Meanwhile, when proceeding from step S67 to step S68, the examination code estimation function 54 determines one examination code specified by the user via the input interface 41 by the button 82 as the examination code presumed to be associated with the examination information.

According to the above procedure, by using the examination information obtained from the examination record, it is possible to estimate the examination code corresponding to the examination based on the examination code table 61.

In addition, according to the procedure shown in FIG. 5, when the examination code is included in the examination order and the order code and the estimated code are different from each other, the medical information processing apparatus and the X-ray CT apparatus 1 according to the present embodiment can notify the user of the information to that effect. For example, in step S3 of FIG. 4, when the user wrongly sets the imaging conditions, or intentionally sets the imaging conditions different from the examination order, the order code and the estimated code may be different. Even in such a case, according to the medical information processing apparatus and the X-ray CT apparatus 1 according to the present embodiment, by using the examination code confirmation notification image 81 (see FIG. 7), the user can confirm whether or not the examination is executed under the correct imaging conditions and can confirm the appropriate examination code that is presumed to be associated with the record of the executed examination.

According to at least one of the above-described embodiments, it is possible to support so as to easily associate the examination code with the examination information of the executed examination.

The processing circuitry 45 in the above-described embodiments is an example of the processing circuitry described in the claims.

In addition, the term “processor” used in the explanation in the above-described embodiments, for instance, refer to circuitry such as dedicated or general purpose CPUs (Central Processing Units), dedicated or general-purpose GPUs (Graphics Processing Units), or ASICs (Application Specific Integrated Circuits), programmable logic devices including SPLDs (Simple Programmable Logic Devices), CPLDs (Complex Programmable Logic Devices), and FPGAs (Field Programmable Gate Arrays), and the like. The processor implements various types of functions by reading out and executing programs stored in the memory circuitry.

In addition, instead of storing programs in the memory circuitry, the programs may be directly incorporated into the circuitry of the processor. In this case, the processor implements each function by reading out and executing each program incorporated in its own circuitry. Moreover, although in the above-described embodiments an example is shown in which the processing circuitry configured of a single processor implements every function, the processing circuitry may be configured by combining plural processors independent of each other so that each processor implements each function of the processing circuitry by executing corresponding program. When a plurality of processors are provided for the processing circuitry, the memory medium for storing programs may be individually provided for each processor, or one memory circuitry may collectively store programs corresponding to all the functions of the processors.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus, comprising processing circuitry configured to:
acquire examination information including a plurality of imaging conditions corresponding to an examination performed on an object;
estimate an examination code corresponding to the examination based on the examination information; and
associate the examination code with the examination information.

2. The X-ray CT apparatus according to claim 1, wherein the processing circuitry is configured to:
estimate an imaged region in the examination by performing image processing on a medical image acquired by the examination; and
estimate the examination code corresponding to the examination based on information on the imaged region.

3. The X-ray CT apparatus according to claim 1, wherein the processing circuitry is configured to estimate the examination code corresponding to the examination based on the examination information, by using parameters obtained beforehand by supervised learning using training data consisting of examination information as an input object and an examination code corresponding to examination information as a desired output value.

4. The X-ray CT apparatus according to claim 1, wherein the processing circuitry is configured to acquire a table in which each of examination codes is associated in advance with corresponding each of types of examination information, and estimate the examination code corresponding to the examination based on the table using the examination information.

5. The X-ray CT apparatus according to claim 4, wherein the processing circuitry is configured to:
when an imaged region is included as the examination information associated with the table, estimate the imaged region in the examination by performing image processing on a medical image acquired by the examination; and
estimate the examination code corresponding to the examination based on the table using the estimated imaged region.

6. The X-ray CT apparatus according to claim 4, wherein a memory storing the table is provided outside an own apparatus,
and wherein the processing circuitry is configured to acquire the table from the memory provided outside the own apparatus via a network.

7. The X-ray CT apparatus according to claim 4, wherein information on the exposure dose is further associated with the examination code in the table in advance.

8. The X-ray CT apparatus according to claim 1, wherein the processing circuitry is configured to:
calculate similarity by comparing the plurality of imaging conditions included in the examination information with imaging conditions included in each of a plurality of examination codes; and
estimate the examination code corresponding to the examination based on the similarity.

9. The X-ray CT apparatus according to claim 1, wherein the examination information further includes a type of the object.

10. The X-ray CT apparatus according to claim 1, wherein each of the plurality of imaging conditions included in the examination information includes at least one of an imaging mode, an imaged region, and presence or absence of a contrast agent.

11. The X-ray CT apparatus according to claim 1, wherein the processing circuitry is configured to:
obtain an examination order; and
when the examination code is included in the examination order and when the examination code included in the examination order is different from the examination code estimated by the processing circuitry as corresponding to the examination, notify a user that the estimated examination code is different from the examination code included in the examination order.

12. The X-ray CT apparatus according to claim 11, wherein the processing circuitry is configured to, when the examination code is included in the examination order and when the examination code included in the examination order is different from the estimated examination code, compare the examination information associated in the table with the examination code included in the examination order and the examination information associated in the table with the estimated estimation code, and notify the user of different information as a result of comparison.

13. The X-ray CT apparatus according to claim 11, wherein the processing circuitry is configured to estimate the examination code designated by the user as the examination code corresponding to the examination when the processing circuitry receives designation of the examination code via an input interface by the notified user.

14. A medical information processing apparatus, comprising processing circuitry configured to:
acquire examination information including a plurality of imaging conditions corresponding to an examination performed on an object;
estimate an examination code corresponding to the examination based on the examination information; and
associate the examination code with the examination information.

15. The medical information processing apparatus according to claim 14, wherein the processing circuitry is configured to estimate the examination code corresponding to the examination based on the examination information, by using parameters obtained beforehand by supervised learning using training data consisting of examination information as an input object and an examination code corresponding to examination information as a desired output value.

16. The medical information processing apparatus according to claim 14, wherein the processing circuitry is configured to acquire a table in which each of examination codes is associated in advance with corresponding each of types of examination information, and estimate the examination code corresponding to the examination based on the table using the examination information.

17. The medical information processing apparatus according to claim 14, wherein the processing circuitry is configured to:
when an imaged region is included as the examination information associated with the table, estimate the imaged region in the examination by performing image processing on a medical image acquired by the examination; and
estimate the examination code corresponding to the examination based on the table using the estimated imaged region.
